(19) United States
(12) Patent Application Publication
(10) Pub. No.: US 2012/0245703 A1
(43) Pub. Date: Sep. 27, 2012

(54) COMPOSITE BONE MATERIAL IMPLANT AND METHOD

(76) Inventor: Thomas L. Meredith, Brentwood, TN (US)

(21) Appl. No.: 13/356,646

(22) Filed: Jan. 23, 2012

Related U.S. Application Data
(63) Continuation-in-part of application No. 10/750,995, filed on Jan. 2, 2004, now abandoned.

(51) Int. Cl. A61F 2/28; A61K 31/7004

(52) U.S. Cl. 623/23.51; 514/23

(57) ABSTRACT

The present invention relates to a method of forming a bone composite, comprising: providing bone tissue; grinding said bone tissue to form ground tissue; molding the ground bone tissue into a bone composite; applying a binder to the bone composite; applying a vacuum to the mold, and optionally milling or refining the bone composite to the desired shape. The present invention includes the use of a carbohydrate, water, cyanoacrylate and demineralized bone.

This patent application is a continuation-in-part of U.S. patent application Ser. No. 10/750,995, filed Jan. 2, 2004, the contents of which are expressly incorporated herein by reference.

### FIELD OF THE INVENTION

This invention generally relates to the field of bone composite implants and a method of forming bone composites. The bone composite implants, or osteoimplants, of the present invention may be used in the repair, replacement, and/or augmentation of various portions of animal or human skeletal systems. The bone composite implants of the present invention may be considered load-bearing implants. More particularly, the invention relates to an injectable bone composite which solidifies after implantation into the skeletal system.

### BACKGROUND OF THE INVENTION

The practice of donating and transplanting bone tissue is beginning to form an important part of therapy for a number of ailments involving bone.

FIG. 1 is a three-dimensional diagram showing the appearance of both a cross and a longitudinal section of an example of a bone structure, and shows various components of the bone. Of course, FIG. 1 is not as detailed as possible, and does not feature every element of bone tissue. The purpose of FIG. 1 is only to briefly show some features of natural bone which also occur in the bone composites of the present invention. With respect to FIG. 1, a bone 10 section is shown. Lamellae 15 are shown within the bone cross section. The haversian canals 20 are shown. In the longitudinal section of the drawing, blood vessels 25 are shown in connection with the haversian canals 20. Finally, the marrow portion 30 is shown with blood vessels 25 extending into the marrow portion of the bone.

Tissue grafting of living tissue from the same patient, including bone grafting, is well known in medical science. Tissue such as bone is removed from one part of a body (the donor site) and inserted into tissue in another (the host site) part of the same (or another) body. This method has been desirable in the past because the tissue was believed to be highly osteoconductive. With respect to living bone tissue, it has been desirable in the past to be able to remove a piece of living tissue graft material which is the exact size and shape needed for the host site where it will be implanted, but it has often proved very difficult to achieve this goal.

Until recently, developers of bone transplants and prostheses have believed that it is desirable to maintain graft tissue in a living state during the grafting process. It is relatively undisputed that the use of living tissue in a graft will promote bone healing, but recent surgical experience has shown that healing can be achieved with allografts of non-living bone material which has been processed.

Processing of bone material which does not contain living tissue is becoming more and more important. Non-living bone grafting techniques have been attempted both for autografts and for allografts. The use of autograft bone is where the patient provides the source of the bone, and the use of allograft bone is where another individual of the same species provides the source of the bone.

It is now possible to obtain allograft bone which has been processed to remove all living material which could present a tissue rejection problem or an infection problem. Such processed material retains much of the mineral quality of the original living bone, rendering it more osteoinductive. Moreover, it can be shaped according to known and new methods to attain enhanced structural behavior. In fact spine surgeons express a distinct preference for such materials, and at least one supplier, the Musculoskeletal Transplant Foundation (MTF), has introduced femoral ring allografts for spine surgeries.

In the fabrication of bone transplants, it is observed that bone material which yields to compressive loads at the exterior surfaces without significant degradation of the interior structural properties, such as cancellous or trabecular bone, can be shaped. It is not unusual that reshaping of a graft tissue is necessary to obtain the best possible graft. In particular, bone tissue may be stronger and better able to bear force when it is denser and more compact.

Allograft bone occurs in two basic forms: cancellous bone (also referred to as trabecular bone) and cortical bone. Cortical bone is highly dense and has a compound structure comprised of calcium hydroxyapatite reinforced with collagen fiber. In the present invention, cortical bone tissue is preferred.

Compression allows conversion of larger irregular shapes into the desirable smaller shape, thereby permitting more disparate sources of allograft bone to be used. By compressing bone to a given shape it is possible to configure the allograft to match a preformed donee site prepared by using a shaped cutter to cut a precisely matching cut space. In particular, this method of formation facilitates the formation of match mated surfaces of the implant for the formation of a particular shape for skeletal repair or revision.

For the reasons stated above, in certain embodiments of the present invention, compression is useful as part of the molding step in forming the bone composites of the present invention. However, an advantage of the present invention is that in some embodiments compression is not required, and in some embodiments it is preferred—but at very low pressure when compared to the compression levels of the prior art.

In response to the need for a composite material to make use of bone fragments and bone powder for fabricating implants and prosthetic devices for bone the current inventor developed the present invention. Advantageously, the invention uses carbohydrates with distilled water and cyanoacrylate and demineralized bone so as to provide a customizable bone composite structure.

### SUMMARY OF THE INVENTION

An object of the present invention is to produce a bone tissue composite that is osteoinductive and has excellent strength characteristics, including excellent load-bearing ability.

Another object of the current invention is to provide a composite material utilizing bone powder and/or fragments as well as a method to manufacture and shape the composite into usable implants and/or bone prostheses. In preferred embodiments of the present invention, composite formed from the method of the present invention is of sufficient strength in a body fluid environment to enable the osteoimplant to bear loads.

Another object of the present invention is to provide a method which enables the fabrication of the composites into any size or shape for use as an implant.

Furthermore, it is an object of the present invention to provide a bone composite that is readily received and hosted when received by another mammal. The composite of the present invention allows bone fusion to occur, and the biocompatible and osteoinductive process allows the body to lay down native bone in combination with the implanted bone composite.

Still another object of the present invention is to provide an injectable bone composite capable of being implanted into a bone structure within a species.

Still yet another object of the present invention is to provide an injectable bone composite capable having the capacity to solidify after implantation into a bone structure of a species and maintain a load bearing structural integrity within the bone structure of the species.

More specifically, the present invention relates to a method of forming a bone composite, comprising: providing bone tissue; grinding said bone tissue to form ground tissue; transferring the ground bone tissue into a mold; applying a binder to the bone tissue; applying a vacuum to the mold; and optionally milling or refining the bone composite to the desired shape. Preferably, the bone tissue is substantially cortical bone tissue (i.e., greater than about 40-50%), and preferably, the bone tissue is substantially demineralized (i.e., greater than about 40-50%).

More preferably, the bone tissue is greater than about 50% cortical bone tissue, more preferably in the range of greater than about 50-70% cortical bone tissue, more preferably in the range of greater than about 50-90% cortical bone tissue, more preferably in the range of greater than about 50-95% cortical bone tissue, more preferably 90% cortical bone tissue, and more preferably greater than about 95% cortical bone tissue. The size of the ground bone particles can vary, but typically the particles will range in size from 125 to 850 microns in size.

The molding process of the present invention occurs at from 14.7 psi (atmospheric pressure) to less than about 1,000 psi. Preferably, the above-mentioned occurs at below than 500 psi. Most preferably, the above-mentioned occurs at below about 200 psi.

Another embodiment of the present invention is a method of forming a bone composite, comprising: (i) providing bone tissue; (ii) grinding said bone tissue to form ground bone tissue ranging in size from about 125 microns to about 850 microns; (iii) transferring said ground bone tissue into a mold; (iv) applying a cyanoacrylate binder to the bone tissue; (v) applying a vacuum to the mold; (vi) applying a compressive force of less than 1000 psi to the mold; and (vii) optionally milling or refining the bone composite to the desired shape.

Another embodiment is a bone composite produced by one of the processes of the present invention. This composite is osteoinductive, and comprises ground bone tissue molded to form a desired shape, and a cyanoacrylate binder. The molded bone composite of the present invention further comprises random voids. The voids, discussed further below, aid osteoconductivity.

Another embodiment of the present invention is a method of forming a bone composite comprising; providing dematerialized bone matrix, or bone tissue a n-Butyl Cyanoacrylate, and sterile water into a bone structure within a species in order to implant an injectable bone composite.

These and other embodiments will become apparent in the more detailed disclosure that follows.

### SUMMARY OF THE INVENTION

An optional aspect of the invention includes an injectable bone composite implanted into a bone structure within a species, having a demineralized bone matrix; n-butyl cyanoacrylate; sterilized water; and carbohydrate.

Further optional aspects include having water with a pH factor greater than 7.0.

Even further optional aspects include the water being distilled.

Further optional aspects include the n-Butyl Cyanoacrylate being absent methyl and ethyl.

Yet further optional aspects include the carbohydrate being glucose.

And further optional aspects include the carbohydrate being present from about 0.05% to about 1% of the total weight of the injectable bone composite.

Further optional aspects include the bone matrix being sterile allogeneic ground bone sized less than 1000 microns.

Further optional aspects include the sterile allogeneic ground bone being sized greater than 10 microns.

Yet further optional aspects include the sterile allogeneic ground bone consisting of bone particles sized greater than 125 microns.

And further optional aspects include the sterile allogeneic ground bone being freeze-dried.

Even further optional aspects include the demineralized bone matrix being from about 0.05% to about 1% carbohydrate with the balance of the composite comprising about equal portions of n-Butyl Cyanoacrylate, water and demineralized bone.

And further optional aspects include bone composite implanted into a bone structure within a species, the injectable bone composite having a demineralized bone matrix; n-butyl cyanoacrylate; sterilized water; and glucose.

In further optional aspects the concentration of demineralized bone matrix is from about 0.05% to about 1% glucose with the balance of the composite comprising about equal portions of n-Butyl Cyanoacrylate, water and demineralized bone.

Even further optional aspects include a bone composite where having approximately 1% glucose is more osteoinductive than a bone composite with less than 0.01% glucose.

And further optional aspects include a bone composite where having approximately 1% glucose has less compressive strength than a bone composite with less than 0.01% glucose.

Further optional aspects include a method of forming a bone composite by providing about equal proportions of distilled water, cyanoacrylate, and demineralized bone; choosing whether the bone composite should be more osetoinductive or have greater compressive strength; selecting between the addition of a lower percentage of carbohydrate for a composite with greater compressive strength or a higher percentage of carbohydrate for a composite with greater osetoinductivity; and providing an amount of carbohydrate ranging from about 0.05% of the total weight of the composite to about 1% of the total weight of the composite.

In further optional aspects, the carbohydrate can always be glucose.

### BRIEF DESCRIPTION OF THE DRAWINGS

FIG. 1 shows a 3-dimensional diagram of a cross and longitudinal section of a bone portion. FIG. 1 shows a few of the features that appear in natural bone.

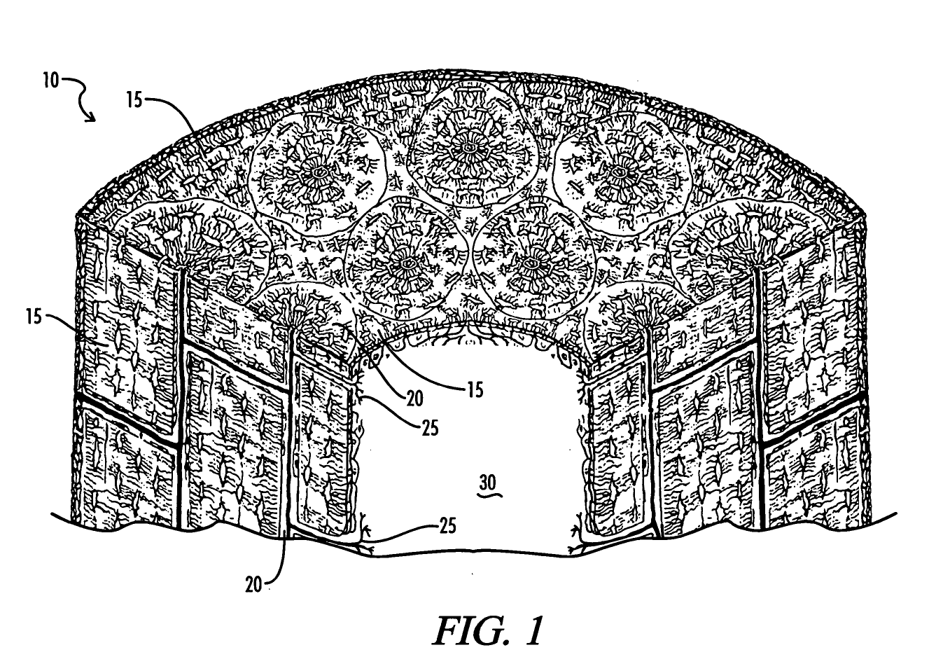

FIG. 2 shows a cross section drawing of an example of a bone composite of the present invention.

FIG. 3 shows magnified photographs examples of bone composite of the present invention.

FIG. 4 also shows magnified photographs of the examples of the bone composite of the present invention.

### DETAILED DESCRIPTION OF THE PREFERRED EMBODIMENTS

The method and composite of the present invention can be used with any mammal, preferably horses and humans, though generally humans. However, it is preferred that donor bone is the same species as recipient bone. That is, preferably human bone is used in making a bone composite that will be used by a human. Preferably the bone tissue is demineralized.

“Demineralized,” as applied to the bone particles used herein, is intended to cover all bone particles that have had some portion of their original mineral content removed by a demineralization process. The bone particles are optionally demineralized in accordance with known and conventional procedures in order to reduce their inorganic mineral content. Demineralization methods remove the inorganic mineral component of bone by employing acid solutions. Such methods are well known in the art, see for example, Reddi et al., Proc. Nat. Acad. Sci. 69, pp 1601-1605 (1972). The strength of the acid solution, the shape of the bone particles and the duration of the demineralization treatment will determine the extent of demineralization. Reference in this regard may be made to Lewandrowski et al., J Biomed Materials Res, 31, pp 365-372 (1996).

As utilized herein, the phrase “superficially demineralized” as applied to the bone particles refers to bone particles possessing at least about 90 weight percent of their original inorganic mineral content. The phrase “partially demineralized” as applied to the bone particles refers to bone particles possessing from about 8 to about 90 weight percent of their original inorganic mineral content, and the phrase “fully demineralized” as applied to the bone particles refers to bone particles possessing less than about 8, preferably less than about 1, weight percent of their original inorganic mineral content. The unmodified term “demineralized” as applied to the bone particles is intended to cover any one or combination of the foregoing types of demineralized bone particles.

The type of mammalian bone that is most plentiful and most preferred as a resource for the composites of the present invention is cortical bone, which is also the form of bone tissue with the greatest compressive strength. As stated above, preferably cortical bone tissue is used to form the composites of the present invention. Also, preferably, the composites are substantially cortical bone tissue. As another preferred embodiment, the composites are above 50% cortical bone tissue, more preferably the bone tissue is greater than about 70%, greater than about 90%, or greater than about 95% cortical bone tissue.

The bone tissue may be ground or pulverized. Pulverized bone can be collected and separated into a number of batches, each batch comprising a different mean particle size. The particle size can vary from fine to coarse. The properties of the composite to be produced can be tailored by choice of particle size. For example, particles in the range of from about 125 to about 850 microns can be used for making bone composites useful for skeletal repair and revision.

The resulting bone powder is placed in a mold and compressed using compression tooling. The measurements of the bone powder (weights and volume) are all predetermined, and one of ordinary skill in the art would understand the measurements to be dependant upon the size and shape of the desired resulting composite to be manufactured.

In a preferred embodiment, the ground bone tissue is hydrated before being place in the mold. Most preferably, the ground bone tissue is hydrated in an amount of about 1 to about 10% (volume), preferably in an amount of about 1 to about 5%. The hydrate is preferably dimineralized water, and is preferably applied by injection, spray bath, or soaking.

The mold may be any commercially mold that has pneumatic or vacuum capabilities. Preferably, the mold is a virgin Teflon®, or polyethylene mold that is contained in a stainless steel envelope. The mold preferably has a stainless steel pneumatic cylinder, vacuum pump, exhaust filtration, and pneumatic silencers.

Typically the input pressure, bore size of the pneumatic cylinder, and vacuum level (inches of Hg based on a standard barometer reading at atmospheric pressure (14.7 psi)) is predetermined and dependent upon the desired size, desired shape, and desired density of the composite to be manufactured. For example, one needs at least the following: Input pressure; bore size of the pneumatic cylinder; vacuum level during CA fill, etc.

The mold preferably will incorporate predetermined number of orifices of a predetermined size, to help assure that the composite will receive evenly distributed pneumatic induced pressure and vacuum flow (Pascal's law). Pascal's Law can be understood in resulting in equally and momentarily retaining the combination of demineralized bone, cyanoacrylate, water and carbohydrate within a vessel that sees a constant >1.0 psig pressure being applied at one end of a special stainless steel, Teflon or polyethylene mold. On the opposite end there can be another opening which is generally screened or filtered that can be subject to an around 28 psig vacuum flow. All of this may be timed for a period of no less than about 30 seconds or more depending upon the size of subjected composite. During this procedure water can remain within the vessel while a significant portion of the water (generally distilled) is vacuumed out in a mostly gaseous state. After a minimum time of about 20 minutes, the composite may in optional embodiments be allowed to dehydrate for 20 minutes or dependant of size for a greater amount of time. By weight, the composite can be void of all 33% water weight and N-butyl cyanoacrylate retained (polymerized) and attached to bone particles as lattice work (small beads) can generally be retained at about less than 20% by weight.

The bone particles of the present invention may be combined with one or more of the biocompatible components set forth in U.S. Pat. No. 6,294,187, incorporated herein by reference. That is, the present invention may be combined with one or more biocompatible components such as wetting agents, biocompatible binders, fillers, fibers, plasticizers, biostatic/biocidal agents, surface active agents, bioactive agents, and the like, prior to, during, or after compressing the bone particle-containing composition. One or more of such components can be combined with the bone particles by any suitable means, e.g., by soaking or immersing the bone particles in a solution or dispersion of the desired component, by physically admixing the bone particles and the desired component, and the like.

At least a binder may be applied to the bone powder. The binder may be applied by an injection, spray, bath, soaking, or layering. Preferably the binder is applied to the bone tissue in the mold, and preferably during a period while the mold is under vacuum. The binder should be biocompatible. Preferably the binder is a cyanoacrylate.

Suitable wetting agents include biocompatible liquids such as water, organic protic solvent, aqueous solution such as physiological saline, concentrated saline solutions, sugar solutions, ionic solutions of any kind, and liquid polyhydroxy compounds such as glycerol and glycerol esters, and mixtures thereof. The use of wetting agents in general is preferred in the practice of the present invention, as they improve handling of bone particles. When employed, wetting agents will typically represent from about 20 to about 80 weight percent of the bone particle-containing composition, calculated prior to compression of the composition. Certain wetting agents such as water can be advantageously removed from the osteoimplant, e.g., by heating and lyophilizing the osteoimplant.

Suitable biocompatible binders include biological adhesives such as fibrin glue, fibrinogen, thrombin, mussel adhesive protein, silk, elastin, collagen, casein, gelatin, albumin, keratin, chitin or chitosan; cyanoacrylates; epoxy-based compounds; dental resin sealants; bioactive glass ceramics (such as apatite-wollastonite), dental resin cements; glass ionomer cements (such as Lonocap® and Inocem® available from lonos Medizinische Produkte GmbH, Greisberg, Germany); gelatin-resorcinol-formaldehyde glues; collagen-based glues; cellulosics such as ethyl cellulose; bioabsorbable polymers such as starches, polylactic acid, polyglycolic acid, polylactic-co-glycolic acid, polydioxanone, polycaprolactone, polycarbonates, polyorthoesters, polyamino acids, polyanhydrides, polyhydroxybutyrate, polyhyroxyvalyrate, poly(propylene glycol-co-fumaric acid), tyrosine-based polycarbonates, pharmaceutical tablet binders (such as Eudragit® binders available from Huls America, Inc.), polyvinylpyrrolidone, cellulose, ethyl cellulose, micro-crystalline cellulose and blends thereof; starch ethylenevinyl alcohols, polycyanoacrylates; polyphosphazenes; nonbioabsorbable polymers such as polyacrylate, polymethyl methacrylate, polytetrafluoroethylene, polyurethane and polyamide; etc. Preferred binders are polyhydroxybutyrate, polyhydroxyvalerate and tyrosine-based polycarbonates. When employed, binders will typically represent from about 5 to about 70 weight percent of the bone particle-containing composition, calculated prior to compression of the composition.

The binder acts as a matrix which binds the bone particles, thus providing coherency in a fluid environment and also improving the mechanical strength of the osteoimplant. Preferably, the binder is a cyanoacrylate binder. More preferably, the cyanoacrylate binder comprises ester chain, N-butyl, or butyl cyanoacrylates. Also, preferably the cyanoacrylate is a long chain cyanoacrylates.

Suitable fillers include graphite, pyrolytic carbon, bioceramics, bone powder, demineralized bone powder, anorganic bone (i.e., bone mineral only, with the organic constituents removed), dentin tooth enamel, aragonite, calcite, nacre, amorphous calcium phosphate, hydroxyapatite, tricalcium phosphate, Bioglass® and other calcium phosphate materials, calcium salts, etc. Preferred fillers are demineralized bone powder and hydroxyapatite. When employed, filler will typically represent from about 5 to about 80 weight percent of the bone particle-containing composition, calculated prior to compression of the composition.

Suitable fibers include carbon fibers, collagen fibers, tendon or ligament derived fibers, keratin, cellulose, hydroxyapatite and other calcium phosphate fibers. When employed, fiber will typically represent from about 5 to about 75 weight percent of the bone particle-containing composition, calculated prior to compression of the composition.

Suitable plasticizers include liquid polyhydroxy compounds such as glycerol, monoacetin, diacetin, etc. Glycerol and aqueous solutions of glycerol are preferred. When employed, plasticizer will typically represent from about 20 to about 80 weight percent of the bone particle-containing composition, calculated prior to compression of the composition.

Suitable biostatic/biocidal agents include antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamicin, povidone, sugars, mucopolysaccharides, etc. Preferred biostatic/biocidal agents are antibiotics. When employed, biostatic/biocidal agent will typically represent from about 10 to about 95 weight percent of the bone particle-containing composition, calculated prior to compression of the composition.

Suitable surface active agents include the biocompatible nonionic, cationic, anionic and amphoteric surfactants. Preferred surface active agents are the nonionic surfactants. When employed, surface active agent will typically represent from about 1 to about 80 weight percent of the bone particle-containing composition, calculated prior to compression of the composition.

Any of a variety of bioactive substances can be incorporated in, or associated with, the bone particles. Thus, one or more bioactive substances can be combined with the bone particles by soaking or immersing the bone particles in a solution or dispersion of the desired bioactive substance(s). Bioactive substances include physiologically or pharmacologically active substances that act locally or systemically in the host.

Bioactive substances which can be readily combined with the bone particles include, e.g., collagen, insoluble collagen derivatives, etc., and soluble solids and/or liquids dissolved therein; antiviricides, particularly those effective against HIV and hepatitis; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamicin, etc.; biocidal/biostatic sugars such as dextran, glucose, etc.; amino acids; peptides; vitamins; inorganic elements; co-factors for protein synthesis; hormones; endocrine tissue or tissue fragments; synthesizers; enzymes such as collagenase, peptidases, oxidases, etc.; polymer cell scaffolds with parenchymal cells; angiogenic agents and polymeric carriers containing such agents; collagen lattices; antigenic agents; cytoskeletal agents; cartilage fragments; living cells such as chondrocytes, bone marrow cells, mesenchymal stem cells, natural extracts, genetically engineered living cells or otherwise modified living cells; DNA delivered by plasmid or viral vectors; tissue transplants; demineralized bone powder; autogenous tissues such as blood, serum, soft tissue, bone marrow, etc.; bioadhesives, bone morphogenic proteins (BMPs); osteoinductive factor; fibronectin (FN); endothelial cell growth factor (ECGF); cementum attachment extracts (CAE); ketanserin; human growth hormone (HGH); animal growth hormones; epidermal growth factor (EGF); interleukin-1 (IL-1); human alpha thrombin; transforming growth factor (TGF-beta); insulin-like growth factor (IGF-1); platelet derived growth factors (PDGF); fibroblast growth factors (FGF, bFGF, etc.); periodontal ligament chemotactic factor (PDLGF); somatotropin; bone digesters; antitumor agents; immuno-suppressants; permeation enhancers, e.g., fatty acid esters such as laureate, myristate and stearate monoesters of polyethylene glycol, enamine derivatives, alpha-keto aldehydes, etc.; and nucleic acids. Preferred bioactive substances are currently bone morphogenic proteins and DNA delivered by plasmid or viral vector. When employed, bioactive substance will typically represent from about 0.1 to about 20 weight percent of the bone particle-containing composition, calculated prior to compression of the composition.

It will be understood by those skilled in the art that the foregoing biocompatible components are not intended to be exhaustive and that other biocompatible components may be admixed with bone particles within the practice of the present invention.

The total amount of such optionally added biocompatible substances will typically range from about 0 to about 95% weight/volume (w/v), preferably from about 1 to about 60% w/v, more preferably from about 5 to about 50% w/v, weight percent of the bone particle-containing composition, based on the weight of the entire composition prior to compression of the composition, with optimum levels being readily determined in a specific case by routine experimentation.

One method of fabricating the bone particle-containing composition which can be advantageously utilized herein involves wetting a quantity of bone particles, of which at least about 60 weight percent preferably constitute elongate bone particles, with a wetting agent as described above to form a composition having the consistency of a slurry or paste. Optionally, the wetting agent can comprise dissolved or admixed therein one or more biocompatible substances such as biocompatible binders, fillers, plasticizers, biostatic/biocidal agents, surface active agents, bioactive substances, etc., as previously described.

Preferred wetting agents for forming the slurry or paste of bone particles include water, liquid polyhydroxy compounds and their esters, and polyhydroxy compounds in combination with water and/or surface active agents, e.g., the Pluronics® series of nonionic surfactants. Water is the most preferred wetting agent for utilization herein. The preferred polyhydroxy compounds possess up to about 12 carbon atoms and, where their esters are concerned, are preferably the monoesters and diesters. Specific polyhydroxy compounds of the foregoing type include glycerol and its monoesters and diesters derived from low molecular weight carboxylic acids, e.g., monoacetin and diacetin (respectively, glycerol monoacetate and glycerol diacetate), ethylene glycol, diethylene glycol, triethylene glycol, 1,2-propanediol, trimethylolethane, trimethylolpropane, pentaerythritol, sorbitol, and the like. Of these, glycerol is especially preferred as it improves the handling characteristics of the bone particles wetted therewith and is biocompatible and easily metabolized. Mixtures of polyhydroxy compounds or esters, e.g., sorbitol dissolved in glycerol, glycerol combined with monoacetin and/or diacetin, etc., are also useful. Where elongate bone particles are employed, some entanglement of the wet bone particles will result. Preferably, excess liquid can be removed from the slurry or paste, e.g., by applying the slurry or paste to a form such as a flat sheet, mesh screen or three-dimensional mold and draining away excess liquid.

Where, in a particular composition, the bone particles have a tendency to quickly or prematurely separate or to otherwise settle out from the slurry or paste such that application of a fairly homogeneous composition is rendered difficult or inconvenient, it can be advantageous to include within the composition a substance whose thixotropic characteristics prevent or reduce this tendency. Thus, e.g., where the wetting agent is water and/or glycerol and separation of bone particles occurs to an excessive extent where a particular application is concerned, a thickener such as a solution of polyvinyl alcohol, polyvinylpyrrolidone, cellulosic ester such as hydroxypropyl methylcellulose, carboxy methylcellulose, pectin, xanthan gum, food-grade texturizing agent, gelatin, dextran, collagen, starch, hydrolyzed polyacrylonitrile, hydrolyzed polyacrylamide, polyelectrolyte such as polyacrylic acid salt, hydrogels, chitosan, other materials that can suspend particles, etc., can be combined with the wetting agent in an amount sufficient to significantly improve the suspension-keeping characteristics of the composition.

The binder may be added in an amount to sufficiently provide a cohesive ground bone composite that can be used in skeletal repair and revisions methods without the ground bone coming apart. Preferably, the binder is present in an amount of from about 5% to about 80% w/v. More preferably, the binder may be present in a range of about 20% to about 66% w/v. More preferably, the binder may be present in an amount of from about 20 to about 50%. Another preferred range of binder is it being present in an amount of from about 15% to about 66% w/v.

Additionally, the particular binder used can be varied according to desired properties. For example, cyanoacrylates can be used as a binder in the production of cortical onlay plates and is preferably present in amount of from 20% to 30%. A binder may also be combined with at least one other binder. The binder is applied by injection, spray, bath, soaking or layering.

The above general ranges in optional embodiments allow one of ordinary skill in the art to create a composite of proper density and mechanical properties and further allows the same basic device to be tailored to individual patients and situations.

As stated above, the preferred binder may be a biocompatible cyanoacrylate. Preferred biocompatible cyanoacrylates include ester chain, N-butyl, and butyl cyanoacrylates. When a cyanoacrylate binder is used, a preferred amount is from about 5 to about 80%, preferably from about 20 to about 66%, more preferably from about 20 to about 50%. The cyanoacrylate binder may be combined with at least one other binder. More specifically, the cyanoacrylate binder described herein may also be a cyanoacrylate-comprising binder.

In addition to the materials described above, at least one other adhesive substance can optionally be used as a matrix to form composite bone material (in combination with or without at least one cyanoacrylate). For example, fibrin is a substance formed by human blood when it clots. Fibrin bonds the platelets together in the formation of, e.g., clots and scabs. Alternatively, fibrin glue can be manufactured. Other biocompatible adhesives can also be used. In addition, there exist a number of biocompatible gels which can be used as a matrix adhesive for holding bone powder together to form a composite.

The vacuum force applied to the mold typically ranges from about 29.9 inches of Hg to about 19.7 inches (based on a standard barometer reading of 29.92 inches of Hg at atmospheric pressure being 0% vacuum. The vacuum force may be about 29.5 inches Hg to about 24 inches Hg.

Preferably, the vacuum force may be applied simultaneous with the injection or spraying of binder. The vacuum force helps distribute the binder throughout the ground bone tissue.

The vacuum force may be applied for a period of 1 second to about 10 minutes. Preferably, the vacuum force is applied for a period of less than about 1 minute. More preferably, the vacuum force is applied for a period of less than about 10 seconds.

In addition, pressure during formation can be tailored to the desired outcome. The pressure used in embodiments of the present invention can range from 14.7 psi to less than 1,000 psi. Lower pressures (i.e., from atmospheric to about 100 psi) can be used to form bone composites useful for skeletal repair and revision. Higher pressures (i.e., from about 100 psi to less than 1,000 psi) can be used to form bone composites useful for applications such as a bone screw, and typical load-bearing composites. Preferably, the compressive force is less than about 200 psi.

In certain embodiments of the present invention, a compressive force can be applied to the composite for a period of about 1 second to about 10 minutes. Also, py the compressive force application period can overlap (in whole or in part) with a vacuum application. That is, the compressive force may begin before a vacuum step is complete. Preferably, the compressive force is applied for a period of less than about one minute.

Yet in further optional embodiments, the compressive force can be applied for even lesser durations in instances where the specific formulation does not require as substantial compressive force.

Following the application of the vacuum (and optional compressive force), the composite may be removed from the mold after a period in which the binder is outgassed. Typically the period is about 30 minutes.

Following removal from the mold, the composite may be shaped into the desired product. Alternatively, if the mold is shaped as the desired product, the composite may be inspected for any out of tolerance measurement or shape. Differences can be corrected in any number of ways, including with a light file, grinding, or milling. The composite can be sterilized and packaged.

As stated above, the process of the present invention in optional aspects can comprise (i) providing bone tissue; (ii) grinding said bone tissue to form ground bone tissue; (iii) transferring said ground bone tissue into a mold; (iv) applying a binder to the bone tissue; (v) applying a vacuum to the mold; and (vi) optionally milling or refining the bone composite to the desired shape. In certain embodiments, the process of the present invention may comprise beginning the binder application at the same time the vacuum application begins. In another embodiment, the binder application may overlap in time (in whole or in part) with the vacuum period. Furthermore, a vacuum period may overlap in time (in whole or in part) with a compression period. Without being bound by theory, pressure and vacuum being applied at the same time helps assure even distribution of bone and binder.

In other embodiments, there may be a second vacuum period.

In a preferred embodiment, the process comprises the following steps: the ground bone tissue is placed in a mold, the vacuum pump is activated as the binder is injected, the pump is deactivated, compression begins, compression ends, a second vacuum period occurs, and the composite is removed from the mold.

Usually, it is desirable to allow the binder (especially a cyanoacrylate binder) to gas-off for a period of about 30 minutes after the molding process. Also, during this period, the wetting agent or hydrate can be allowed to evaporate. To accelerate gas-off and evaporation, the molded composite can be exposed to vacuum.

Furthermore, crosslinking, may be performed in order to improve the strength of the osteoimplant. Such crosslinking of the bone particle-containing composition can be effected by a variety of known methods including chemical reaction, the application of energy such as radiant energy, which includes irradiation by UV light or microwave energy, drying and/or heating and dye-mediated photo-oxidation; dehydrothermal treatment in which water is slowly removed while the bone particles are subjected to a vacuum; and, enzymatic treatment to form chemical linkages at any collagen-collagen interface.

Chemical crosslinking agents include those that contain bifunctional or multifunctional reactive groups, and which react with surface-exposed collagen of adjacent bone particles within the bone particle-containing composition. By reacting with multiple functional groups on the same or different collagen molecules, the chemical crosslinking agent increases the mechanical strength of the osteoimplant.

Chemical crosslinking involves exposing the bone particles presenting surface-exposed collagen to the chemical crosslinking agent, either by contacting bone particles with a solution of the chemical crosslinking agent, or by exposing bone particles to the vapors of the chemical crosslinking agent under conditions appropriate for the particular type of crosslinking reaction. For example, the osteoimplant of this invention can be immersed in a solution of cross-linking agent for a period of time sufficient to allow complete penetration of the solution into the osteoimplant. Crosslinking conditions include an appropriate pH and temperature, and times ranging from minutes to days, depending upon the level of crosslinking desired, and the activity of the chemical crosslinking agent. The resulting osteoimplant may then be washed to remove all leachable traces of the chemical.

In optional embodiments using suitable chemical crosslinking agents they can include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as other heterobifunctional crosslinking agents; hexamethylene diisocyante; sugars, including glucose, will also crosslink collagen.

Glutaraldehyde crosslinked biomaterials may have a tendency to over-calcify in the body. In this situation, should it be deemed necessary, calcification-controlling agents can be used with aldehyde crosslinking agents. These calcification-controlling agents include dimethyl sulfoxide (DMSO), surfactants, diphosphonates, aminooleic acid, and metallic ions, for example ions of iron and aluminum. The concentrations of these calcification-controlling agents can be determined by routine experimentation by those skilled in the art.

When enzymatic treatment is employed, useful enzymes include those known in the art which are capable of catalyzing crosslinking reactions on proteins or peptides, preferably collagen molecules, e.g., transglutaminase.

Formation of chemical linkages can also be accomplished by the application of energy. One way to form chemical linkages by application of energy is to use methods known to form highly reactive oxygen ions generated from atmospheric gas, which in turn, promote oxygen crosslinks between surface-exposed collagen. Such methods include using energy in the form of ultraviolet light, microwave energy and the like. Another method utilizing the application of energy is a process known as dye-mediated photo-oxidation in which a chemical dye under the action of visible light is used to crosslink surface-exposed collagen.

Another method for the formation of chemical linkages is by dehydrothermal treatment which uses combined heat and the slow removal of water, preferably under vacuum, to achieve crosslinking of bone particles. The process involves chemically combining a hydroxy group from a functional group of one collagen molecule and a hydrogen ion from a functional group of another collagen molecule reacting to form water which is then removed resulting in the formation of a bond between the collagen molecules.

Optional embodiments of the present invention include a method of forming a bone composite, comprising (i) providing bone tissue; (ii) grinding said bone tissue to form ground bone tissue ranging in size from about 125 microns to about 850 microns; (iii) transferring said ground bone tissue into a mold; (iv) applying a cyanoacrylate binder to the bone tissue; (v) applying a vacuum to the mold; (vi) applying a compressive force of less than 1000 psi to the mold; (vii) providing a carbohydrate and water; and (viii) optionally milling or refining the bone composite to the desired shape. In yet further optional embodiments, the last step may be left off where the bone composite is an injectable bone composite.

In this embodiment, the bone tissue may be substantially cortical bone tissue, and may be substantially demineralized. In optional embodiments, the bone tissue is greater than about 90% cortical bone tissue. Furthermore, a vacuum force of about 20 Hg to about 25 Hg may be applied for up to about 1 minute. The compressive force can be for about a period of about 1 second to about 10 minutes and/or is less than 200 psi.

In this embodiment, (v) and (vi) may overlap in time; (iv) and (v) may overlap in time; (v) may be complete before (vi) is complete; and the method may comprise a second application of a vacuum after (vi) is complete.

In yet further embodiments, the osteoinductive bone tissue composite that comprises ground bone tissue can be molded to form a desired shape; and a cyanoacrylate binder. Furthermore, the composite can comprise random “voids”. The voids are spaces between adjacent bone particles, and are present both at the surface of a composite as well as within the interior of the composite. These voids or spaces vary in size and shape and have a width of up to about 1,000 microns. Preferably the width of the void is from about 50-700 microns, more preferably from about 200-500 microns.

Preferably, the voids are present from about 5% to 50% (by volume of the composite). More preferably, the voids are present from about 15% to 35% (by volume), and more preferably, the voids are present in an about of about 25% (by volume).

The voids can appear on the surface area of the composite as well. The presence of the voids on the surface area aids osteoconductivity. Thus, the composite of the present invention can be said as having an osteoinductive surface. A comparison of observations of a cut surface of a composite of the present invention and a cast surface of the present invention would show similar characteristics with respect to the voids.

The voids exist as a result of the process of the present invention, and their existence promote osteoconductivity of the composite. Without being bound by theory, the voids promote osteoconductivity because an influx of undifferentiated mesenchymal cells normally found within osseous structures as well as undifferentiated cells that migrate to the repair site to fill the voids. The action of the osteoinductive properties of the composite induce the undifferentiated cells to differentiate into bone-forming cells that both form bone within the voids as well as remodel the bone particles of the composite matrix into living host bone.

Many voids can be interconnected one to the other, forming canals, channels, or tunnels that run throughout the composite. These canals are similar to haversian canals found in natural bone. The canals vary in size in shape, but typically have a width of about 10 to 500 microns, preferably from 100 to 200 microns.

The canals and voids work together to give the composite a preferred histo-anatomical structure that is similar to natural bone.

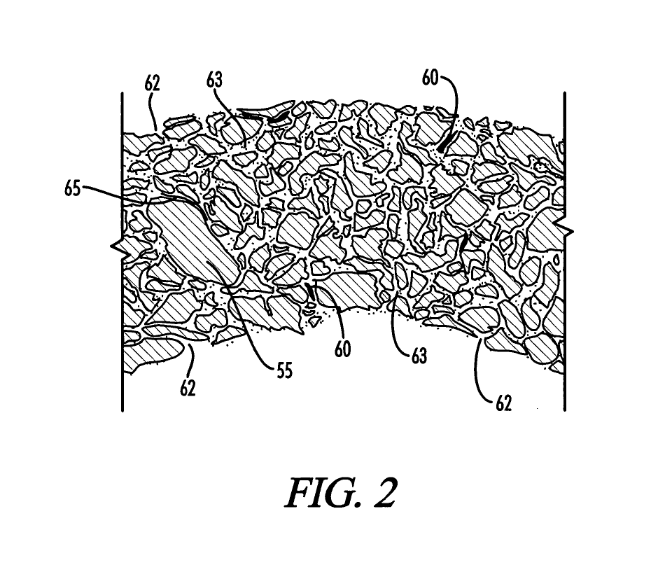

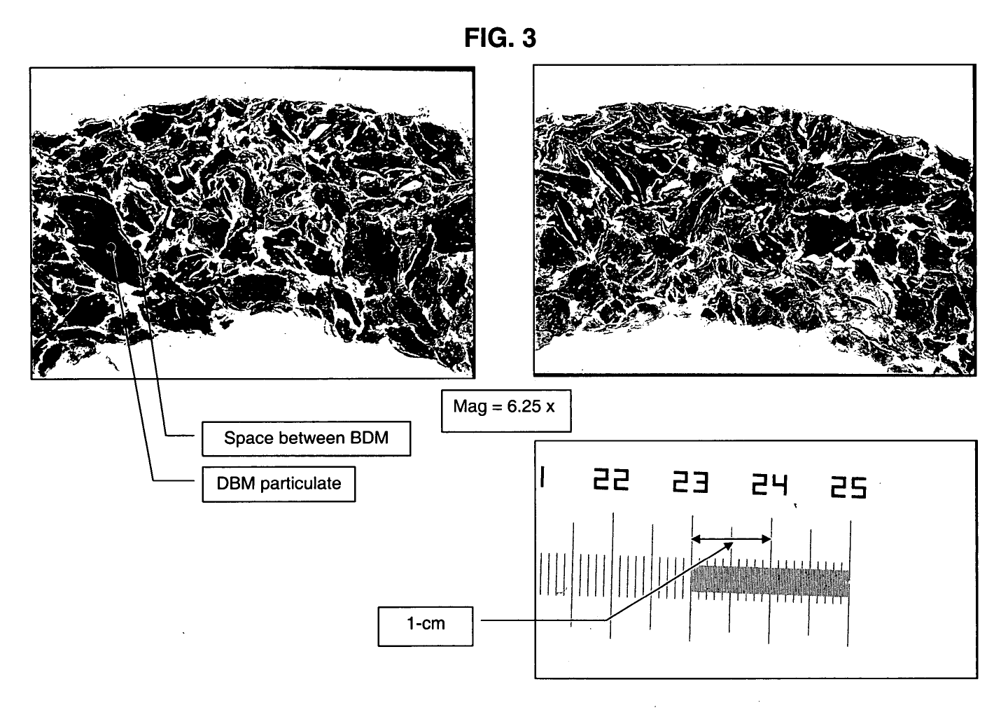

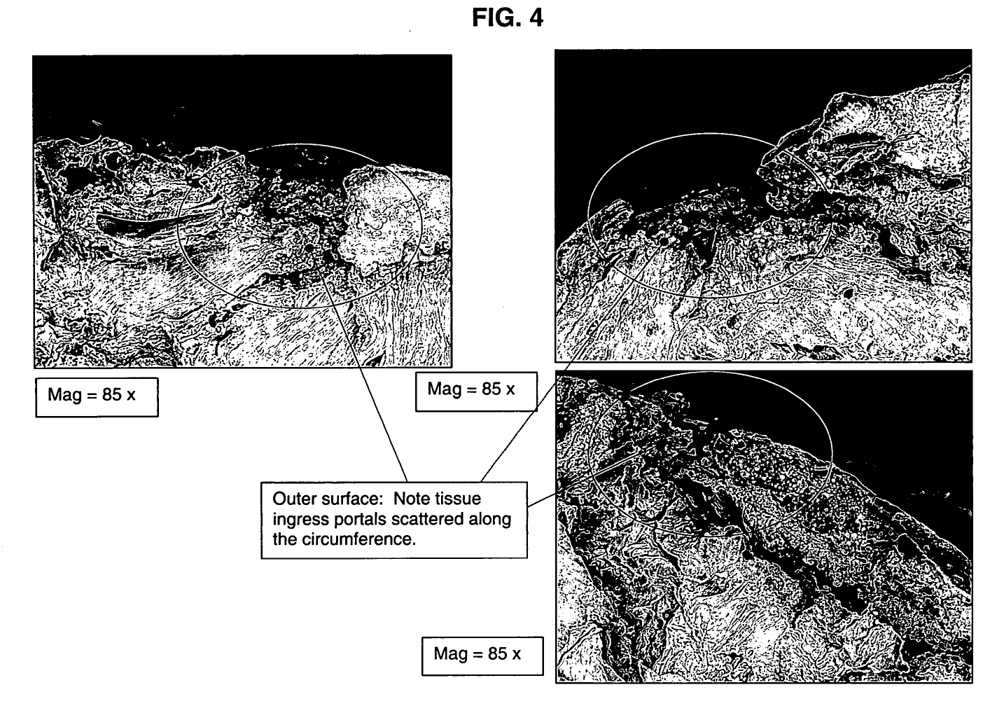

Now turning to the remaining drawings, FIG. 2 is a cross-section of an embodiment of a composite 50 of the present invention. The bone particles 55 are bordered in places by voids 60. The voids 60 join to form canals 65. Also shown in FIG. 2 are surface voids 62. FIG. 3 is a magnified (6.25×) photograph of a composite of the present invention. FIG. 4 is a magnified (85×) photograph of a composite of the present invention. The voids, canals, and bone particles described herein are visible.

As stated above, the composite of the present invention may be formed into a bone pin, screw, sheet, plate, disk, cylinder or prosthesis. In many applications, the general shape can be formed in a specially-shaped mold, and then fine-tuned my milling, etc. after the molding process is complete. One of ordinary skill in the art would recognize many other beneficial uses for the composite of the present invention.

Of course, one of ordinary skill in the art would further recognize that the composite of the present invention may be molded and then later machined, milled, refined, or shaped by any suitable mechanical shaping means. Computerized modeling can, for example, be employed to provide an intricately-shaped composite which is custom-fitted to the bone repair site with great precision.

The following examples are intended to be for illustrative purposes and do not limit the spirit and scope of the present invention.

### Example 1

This example shows a process of making a tubular or cylindrical composite with a 2 cm OD (outside diameter) and 1.4 cm ID (inside diameter) by 2 cm in length, and 3 mm wall.

Cortical human bone is cleaned and ground into particles varying in size from 125 microns to 850 microns. The ground bone is demineralized, providing deminieralized bone matrix (DBM).

2.00 gms is measured, then hydrated with sterile water to a weight of 2.558 gms (0.558 H₂O). The weighed DBM is then inserted into the cylindrical cavity of a Teflon® mold and manually compacted with a force of 0.5 pounds. One (1) cc of special blended N Butyl Cyanoacrylate (CA) is then injected with a #18 gauge needle into the DBM at the outer edge of the cylindrical shape, and at the same time a vacuum of 28″ hg is applied from the bottom end of mold. Two (2) cc of same N Butyl Cyanoacrylate is then injected with a #18 gauge needle into the center core of the Teflon mold. The total weight of the bone composite is now 3.361 gms. This second injection occurs during application with the vacuum force. After the second injection, a maintained pneumatic force of 100 pounds is applied to the DBM with a maintained pneumatically generated vacuum of 28″ Hg for 10 seconds.

Finally, the mold with the DBM composite is allowed to rest for a period of 30 minutes. When the 30-minute mold rest time is complete. Mold is manually dismantled and DBM bone composite is removed, placed on a Teflon mandrel that matches the I.D. of the tubular shaped DBM bone composite. Then allowed to gas-off for 30 minutes. Weight of composite is now 2.945 grams. The tubular shaped DBM bone composite is trimmed with a sharp instrument while still on mandrel, assuring proper outside dimensions. Mandrel assures inside dimensions and maintains inside dimensions as composite tries to contract. The tubular composite is found to be of good quality with even bonding throughout, may now be clean packaged and sterilized later.

### Example 2

Example 1 was repeated, to obtain the samples discussed below.

The densities of the samples were measured using physical density estimates and the water displacement method. Physical estimates measures a volume based upon physical measurement of the sample dimensions (height×width) and the dry weight of the sample. In this example, the samples were prepared by drying the sample in an oven at 110 C overnight. In the Water displacement method (ASTM D-792) the volume of water displaced is measured and the weight of the sample after drying is measured.

Density estimates based upon these two methods for five ART samples is presented in Table 1.

TABLE 1

| | Measured Density | | |
|---|---|---|---|
| | Water Displacement | | |
| | density estimate, | | |
| Sample ID | gms/cc | | Physical Density Estimate, gms/cc |
| Human - 1 - A | 1.12 | 1.12 | 1.00 |
| Human - 1 - B | 0.96 | 0.98 | 0.86 |
| Rabbit - 3 | 0.96 | 1.07 | 0.96 |
| Rabbit - 4 | 1.09 | 1.07 | 0.83 |
| Rabbit - 5 | 1.07 | 1.07 | 0.91 |
| Mean | 1.04 | 1.062 | 0.912 |

1.051

The weight loss on drying is presented in Table 2. The weight loss on drying is a combination of water losses from evaporation of the water trapped within the spaces within the sample as well as the water contained within the DBM material.

TABLE 2

| | Wet | Dry | | Percent |
|---|---|---|---|---|
| | weight, | Weight, | Weight loss on | weight loss |
| Sample ID | gms | gms | drying, gms | on drying |
| Human - 1 - A | 1.1235 | 0.7568 | 0.3667 | 32.64% |
| Human - 1 - B | 1.2309 | 0.7932 | 0.4377 | 35.56% |
| Rabbit - 3 | 1.2999 | 0.8184 | 0.4815 | 37.04% |
| Rabbit - 4 | 1.152 | 0.6846 | 0.4674 | 40.57% |
| Rabbit - 5 | 1.1853 | 0.7489 | 0.4364 | 36.82% |
| Mean | 1.19832 | 0.76038 | 0.43794 | 36.53% |

In another embodiment of the current invention, an injectable bone composite is disclosed. The injectable bone composite consists essentially of demineralized bone matrix, n-Butyl Cyanoacrylate, sterilized water, and a carbohydrate. The injectable bone composite can be designed to be implanted into a bone structure within a species. For example, the injectable bone composite can be inserted into a location along the skeletal system of an animal or human. Ideally this location is a fissure or void within the bone structure or skeleton system in need of support.

The injectable bone composite is designed to solidify, or set within the bone structure, or skeletal system. After solidification of the injectable bone composite, the location to which the injectable bone composite has been inserted will experience increased structural integrity due to the weight bearing and substantially rigid characteristics of the solidified injectable bone composite. The injectable bone composite is designed to match the structural integrity of the surrounding bone structure within the species to which the injectable bone composite is implanted.

Preferably the n-Butyl Cyanoacrylate is absent methyl and ethyl. Also the sterilized water includes a pH factor greater than or equal to 7.0. In a most preferred embodiment the sterilized water has a pH value greater than 7.0 and has been distilled for purification purposes.

The demineralized bone matrix is generally comprised of sterile allogeneic ground bone. The sterile allogeneic ground bone preferably consists of bone particles that are sized less than 1,000 microns but greater than 10 microns. In a most preferred embodiment the sterile allogeneic ground bone consists of bone particles sized between 125 microns to 850 microns. Preferably, sterile allogeneic ground bone is provided in a freeze-dried state.

The injectable bone composite preferably includes substantially equal concentrations of demineralized bone matrix, n-Butyl Cyanoacrylate, water, and then a carbohydrate present from about 0.05% to about 1% by weight In other optional embodiments, the cyanoacrylate and deminerialized bone can each make up a third of the composition with the carbohydrate and water accounting from the other third of the amount. Thus, one optional combination may be about 33% bone matrix, about 33% cyanoacrylate, about 33% water, and about 1% carbohydrate. The concentrations can vary slightly and still result in the desired effect of an injectable bone composite that solidifies upon an injection into a species and maintains that solidification after injection into the species.

In optional embodiments, the injectable bone composite can be designed to solidify upon the mixing of the n-Butyl Cyanoacrylate binder with the sterilized water. As such the combination of this binder and the sterilized water with the demineralized bone matrix and carbohydrate within a bone structure can provide structural integrity to the location in which the injected bone composite is added. The injectable bone composite can be designed to maintain the solidification after the implementation of the injectable bone composite is complete. As such the injectable bone composite can resist deterioration once implanted into the species.

As for additional embodiments when carbohydrates, for example glucose, are added to the composition of 33% distilled water; when added to the mixture of n-Butyl cyanoacrylate and deminerialized bone matrix, the carbohydrate will cause various percentages of non-polymerization to occur. As this happens, more non-polymerized cyanoacrylate and water can be discharged via vacuum pressure at the bottom of a vessel or composite mold, causing the composite to be less structural in compression as well as leaving the demineralized bone matrix particles to be more osteoinductive than when a carbohydrate is not injected into a composite. Thus this can advantageously enable a surgeon to inject this formula into skeletal segment that does not necessarily need a compressive strength of >4 to 5,000 psi while at the same time providing a patient a better chance to heal quicker than normal.

### Example 3

After molding 5 composites of distilled water, dextrose, n-butyl cyanoacrylate and demineralized bone matrix, the composites are allowed to dehydrate in a sealed capsule until high pressure equipment can be assembled for compressive (Force) strength testing.

Units 1 thru 5 include various levels of Distilled Water (DW) to various mixtures of (DC) Dextrose/Carbohydrates for 33%. Plus 33% n-Butyl CA and 33% DBM.

| | | | | | | Force Avg. |
|---|---|---|---|---|---|---|
| | DW % | | DC % | CA % | DBM % | (Total collapse) |
| Unit 1: | 50 | / | 50 | 33 | 33 | 410 psig |
| Unit 2: | 60 | / | 40 | 33 | 33 | 546 psig |
| Unit 3: | 70 | / | 30 | 33 | 33 | 682 psig |
| Unit 4: | 80 | / | 20 | 33 | 33 | 956 psig |
| Unit 5: | 90 | / | 10 | 33 | 33 | 1,230 psig |

Compression tests without the use of dextrose generally have a force average for collapse of 5000 psig, about the same as that of cancellous bone. As is illustrated, the increasing amounts of carbohydrates resulted in a decrease in strength of the composite. However, the increasing amounts of carbohydrates generally provided for greater osteoinductivity.

As such, one can tailor the composition for either strength or for osteoinductivity, based upon the amount of carbohydrate present within the composite. By using less carbohydrate, the composite is less osteoinductive whereas greater carbohydrate percentages cause lesser strength but greater osteoinductivity.

While glucose is often used, other carbohydrates may possibly be used as well. Generally, the carbohydrates are present from about 0.01% to about 1% though in some embodiments may be present in lesser or greater amounts. Varying such amounts of carbohydrates results in various levels of osteoinductive properties. Carbohydrates can be understood as not allowing as much polymerization to take place. In other words; the demineralized bone particles may not totally envelope bone particles, creating larger voids within the composite for osteoclast and osteoblast activity to take place.

Further optional embodiments include replacement products molded in this manner which can include cortical struts used as an onlay for fracture repair, cortical matchsticks for cervical spine fusion, cortical and unicortical dowels for cervical and lumbar spine fusion, femur/fibula/tibia shaft segments for spine fusion and inter-body fracture repair, humerus shaft segments tumor defects or fractured and failed prosthesis, and a multitude of replacement bone segments now being processed with use of donated allograft bone.

In optional embodiments in the method of forming the composite, the pressure is not on a singular open ended vessel of fluid. Rather, such optional embodiments may include pre-packing manually or via injection hydrated (Carbohydrate and distilled water) DBM into an open ended vessel. Once packed the bottom side of a mold or vessel may have a plug with multi drilled orifices placed into the bottom. Prior to the plug being placed into the bottom a steel screen (generally a 100 micron screen) can be put into place at the bottom. From here the cyanoacrylate can be injected into the top area of the mold at about ⅓rd by weight volume, after the cyanoacrylate has been injected, about 28″ HG vacuum can be applied at the bottom while a manual force of about 1 to 10 psig is at the same time applied to the top side. This pressure and vacuum can vary dependent upon the size of the composite being molded for, generally for a period of from about 10 to 60 seconds. During this period of time the injected cyanoacrylate, in accordance with Pascal's law, be evenly distributed throughout the open ended vessel housing pre-packed and hydrated bone matrix. When the initial time period is complete, the composite can then removed from the open ended vessel and allowed to dehydrate and further gas off to a final composite volume by weight of about greater than 33% to about less than 43% leaving nothing but demineralized bone matrix attached by a lattice work of cyanoacrylate polymer and likely some carbohydrate.

Also included is a method of implanting an injectable bone composite into a species. The method includes mixing demineralized bone matrix, n-Butyl Cyanoacrylate, carbohydrate and water within the bone structure of a species during implantation of the combined elements into the species. The demineralized bone matrix can be mixed with, or hydrated by, the water prior to injection of those two combined elements into the species. Additionally, the carbohydrate can be mixed with the water as well. Alternately, the demineralized bone matrix can be mixed with the n-Butyl Cyanoacrylate prior to injection into the species. In either case, the elements are mixed with the third element during the implantation process.

The combination of water with n-Butyl Cyanoacrylate begins a solidification process of the composite. The addition of the demineralized bone matrix provides bone structure with which substantial rigidity can be realized within the void in the skeletal system with which the injectable bone composite is implanted. The carbohydrates are added so as to provide a custom control of the structural stability/osteoinductivity of the composite.

The feature of the sterile water having a pH factor greater than 7.0 promotes osteoinductivity, or remodeling, of the species bone within the bone structure to which the injectable bone composite is implanted. If the water has a pH factor of less than 7.0, a deterring, or masking, effect will occur. This masking effect will occur by an absorbing, or coating, of the morphogenetic proteins. This absorption does not facilitate osteoinductivity. Alternatively stated, the proteins within the demineralized bone matrix will be masked by water with a pH factor of less than 7.0 and will not properly interact with the natural bone within a species to promote osteoinductivity.

The presence of lipoid and blood within the species can aid the solidification and instantaneous setting of the injectable bone composite within the species.

The presence of increased pressure during the implantation of the injectable bone composite into the species can increase the uniform application of the injectable bone composite. For example, increased pressure by injection of the injectable bone composite or increased pressure by vacuum on the injectable bone composite can facilitate a better application of the injectable bone composite within the bone structure of the species.

The following are examples of methods of making and or implanting the injectable bone composite into a species.

### Example 1

The location for the addition of the injectable bone composite within the bone structure of the species is prepared. Normally this is accomplished by preparing a void or opening within the bone structure that is in need of structural support. Sterile, allogeneic ground bone is provided with a particle size ranging from 10 microns to 1,000 microns. This sterile allogeneic ground bone is then hydrated and surgically placed, or packed, in the prepared bone void. Pressure ranging from an atmospheric pressure of 14.7 pounds per square inch to 100 pounds per square inch maybe utilized to supply the preferred amount of ground bone into the bone void.

Next a preferred amount in n-Butyl Cyanoacrylate is then introduced into the hydrated allogeneic bone. The n-Butyl Cyanoacrylate is introduced by injection, pouring, or by being subjected into a vacuum of up to 28 inches of mercury. The hydrated allogeneic ground bone in combination with the n-Butyl Cyanoacrylate will set up a lattice type structure that is load bearing and substantially rigid.

### Example 2

The location within the skeleton system that needs additional structural support is prepared. An apparatus holding demineralized bone matrix, n-Butyl Cyanoacrylate, and sterilized water in segregation can be used to apply these three elements to that bone location within the structural body of the species. The use of filtered dry compressed air or bottled nitrogen can be used with the apparatus to inject the three elements in three equal parts to the skeleton area.

Alternately, the location within the skeleton body that needs the additional structural support can be prepared by boring a entry hole within the skeletal body and boring an exit opening opposite the entry hole. The apparatus that segregates the three elements can then approach the boring entry hole opening and a vacuum attached to the exit boring can draw one equal part from the apparatus into the specific skeleton location.

The use of pressure, and or a vacuum, facilitates the injected media, the bone matrix, n-Butyl Cyanoacrylate, carbohydrate and sterilized water, to flow evenly throughout the void area of the skeletal body. The almost immediate solidification and setting of the injectable bone composite fills the voided skeleton bone area with structural integrity.

Also as the remodeling within the skeleton system occurs, the structural integrity of the filled void area will substantially match the structural integrity of that bone of the skeleton structure as a whole.

The physical shape in which the injected bone composite solidifies to can be controlled through the use of strategically placed shaping objects. These shaping objects can be such things as enforcement pins, rods, cages, wire frames, etc. and can be manufactured out of titanium, stainless steel, and other suitable material.

The use of the injectable bone composite has the capability of deterring or possibly stopping the advancement of bone deterioration and the disease of osteoporosis. This increases the quality of life of the species of which the injectable bone composite was implanted. Also the injectable bone composite can provide swift healing and structural integrity for the fixation of a broken bone within the skeleton system. This would substantially reduce recovery times in which the species could resume activities.

From the foregoing description of the present invention, those skilled in the art will perceive improvements, changes and modifications, and understand that the specific details shown herein are merely illustrative. Such changes, modifications, and improvements do not depart from the spirit and scope of the following claims.

What is claimed is:

1. An injectable bone composite implanted into a bone structure within a species, the injectable bone composite consisting essentially of:
a demineralized bone matrix;
n-butyl cyanoacrylate;
sterilized water; and
carbohydrate.

2. The injectable bone composite of claim 1, wherein the water includes a pH factor greater than 7.0.

3. The injectable bone composite of claim 2, wherein the water is distilled.

4. The injectable bone composite of claim 1, wherein the n-Butyl Cyanoacrylate is absent methyl and ethyl.

5. The injectable bone composite of claim 1, wherein the carbohydrate comprises glucose.

6. The injectable bone composite of claim 1, wherein the carbohydrate is from about 0.05% to about 1% of the total weight of the injectable bone composite.

7. The injectable bone composite of claim 1, wherein the bone matrix comprises sterile allogeneic ground bone sized less than 1000 microns.

8. The injectable bone composite of claim 7, wherein the sterile allogeneic ground bone consists of bone particles sized greater than 10 microns.

9. The injectable bone composite of claim 7, wherein the sterile allogeneic ground bone consists of bone particles sized greater than 125 microns.

10. The injectable bone composite of claim 6, wherein the sterile allogeneic ground bone is freeze-dried.

11. The injectable bone composite of claim 1, wherein the concentration of demineralized bone matrix comprises from about 0.05% to about 1% carbohydrate with the balance of the composite comprising about equal portions of n-Butyl Cyanoacrylate, water and demineralized bone.

12. An injectable bone composite implanted into a bone structure within a species, the injectable bone composite consisting essentially of:
a demineralized bone matrix;
n-butyl cyanoacrylate;
sterilized water; and
glucose.

13. The injectable bone composite of claim 12, wherein the concentration of demineralized bone matrix comprises from about 0.05% to about 1% glucose with the balance of the composite comprising about equal portions of n-Butyl Cyanoacrylate, water and demineralized bone.

14. The method of claim 13, wherein a bone composite with approximately 1% glucose is more osteoinductive than a bone composite with less than 0.01% glucose.

15. The method of claim 13, wherein a bone composite with approximately 1% glucose has less compressive strength than a bone composite with less than 0.01% glucose.

16. A method of forming a bone composite comprising the steps of:
Providing about equal proportions of distilled water, cyanoacrylate, and demineralized bone;
choosing whether the bone composite should be more osetoinductive or have greater compressive strength;
selecting between the addition of a lower percentage of carbohydrate for a composite with greater compressive strength or a higher percentage of carbohydrate for a composite with greater osetoinductivity; and
providing an amount of carbohydrate ranging from about 0.05% of the total weight of the composite to about 1% of the total weight of the composite,

17. The method of claim 16, wherein the carbohydrate is glucose.
